(19) United States
(12) Patent Application Publication
(10) Pub. No.: US 2019/0365255 A1
(43) Pub. Date: Dec. 5, 2019

(54) BIOLOGICAL INFORMATION MEASURING APPARATUS AND METHOD AND PROGRAM USING THE SAME

(71) Applicants: OMRON CORPORATION, Kyoto (JP); OMRON HEALTHCARE CO., LTD., Kyoto (JP)

(72) Inventors: Tsuyoshi KITAGAWA, Kyoto (JP); Shingo YAMASHITA, Kyoto (JP); Daizo OKA, Kyoto (JP)

(21) Appl. No.: 16/543,975

(22) Filed: Aug. 19, 2019

Related U.S. Application Data
(63) Continuation of application No. PCT/JP2018/009562, filed on Mar. 12, 2018.

(30) Foreign Application Priority Data
Mar. 15, 2017 (JP) 2017-050623

(51) Int. Cl. A61B 5/021; A61B 5/00; A61B 5/022

(52) U.S. Cl. CPC A61B 5/02108; A61B 5/0002; A61B 5/6801; A61B 2562/0219; A61B 2560/0223; A61B 2560/0257; A61B 5/022

(57) ABSTRACT

According to one embodiment, an apparatus includes a sensing apparatus and a calibration device. The sensing apparatus includes: a calculator calculating first information on a position of the sensing apparatus. The calibration device includes: a measuring device intermittently measuring first biological information; a receiver receiving the first information; a calculator calculating second information on a position of the calibration device; a determination unit determining whether or not the sensing apparatus and the calibration device are worn on an identical biological site based on the first and second information; and a calculator calibrating the pulse wave based on the first biological information and calculating second biological information based on the calibrated pulse wave.

### CROSS-REFERENCE TO RELATED APPLICATIONS

This application is a Continuation Application of PCT Application No. PCT/JP2018/009562, filed Mar. 12, 2018 and based upon and claiming the benefit of priority from Japanese Patent Application No. 2017-050623, filed Mar. 15, 2017, the entire contents of all of which are incorporated herein by reference.

### FIELD

The present invention relates to a biological information measuring apparatus for continuously measuring biological information, and a method and a program using the same.

### BACKGROUND

With the advances in sensor technology that have allowed high-performance sensors to be readily available, application of biological information to the treatment for early detection of abnormalities in the body has been increasingly gaining medical importance.

A biological information measuring apparatus is known that is capable of measuring biological information, such as the pulse and the blood pressure, using information detected by a pressure sensor that is in direct contact with a biological site, through which an artery, such as the radial artery at the wrist, passes (see, for example, Jpn. Pat. Appln. KOKAI Publication No. 2004-113368).

The blood pressure measuring apparatus described in Jpn. Pat. Appln. KOKAI Publication No. 2004-113368 uses a cuff to calculate the blood pressure value at a biological site which is different from the site with which the pressure sensor is to be in contact, and generates calibration data from the calculated blood pressure value. By calibrating the pressure pulse wave detected by the pressure sensor using the generated calibration data, the blood pressure value is calculated beat by beat.

However, the blood pressure measuring apparatus described in Jpn. Pat. Appln. KOKAI Publication No. 2004-113368 is large in scale, making it difficult to improve the precision in measurement. Moreover, such a blood pressure measuring apparatus is intended to be operated in a limited environment by a specific person, making it difficult for use in routine care or at home. Furthermore, such a blood pressure measuring apparatus inconveniently requires a large amount of tubing and cabling, making it impractical for use on a daily basis or during sleep.

### SUMMARY

According to a first aspect of the present invention, a biological information measuring apparatus includes a sensing apparatus and a calibration device, the calibration device including: a measuring device that intermittently measures first biological information; a calibration position calculator that calculates calibration position information on a position of the calibration device; and a transmitter that transmits the calibration position information and data including the first biological information to the sensing apparatus, the sensing apparatus including: a receiver that receives the first biological information and the calibration position information; a detector that detects a pulse wave continuously in time; a calculator that calibrates the pulse wave based on the first biological information and calculates second biological information based on the calibrated pulse wave; a sensor position calculator that calculates sensor position information on a position of the sensing apparatus; and a wear-determination unit that determines whether or not the sensing apparatus and the calibration device are being worn on an identical biological site based on the sensor position information and the calibration position information.

According to a second aspect of the present invention, each of the sensor position calculator and the calibration position calculator includes an acceleration sensor or an atmospheric pressure sensor.

According to a third aspect of the present invention, the wear-determination unit determines that the sensing apparatus and the calibration device are being worn on an identical biological site, if a difference in acceleration of the acceleration sensor between the sensor position calculator and the calibration position calculator during a certain period of time is equal to or below a first threshold value, and if a difference in atmospheric pressure of the atmospheric pressure sensor between the sensor position calculator and the calibration position calculator during the certain period of time is equal to or below a second threshold value.

According to a fourth aspect of the present invention, the sensing apparatus further includes a determination unit that determines whether or not a difference between an amplitude value of a pulse wave detected by the detector before or after a period of time during which the calibration device measures the first biological information, and a minimum amplitude value of the pulse wave detected by the detector during the period of time is equal to or greater than a third threshold value, and the wear-determination unit makes a notification of incorrect manner of wearing if the difference is determined as being smaller than the third threshold value.

According to a fifth aspect of the present invention, the calibration device further includes a cuff that is being worn on a living body during measurement of the first biological information by the measuring device, and configured to expand and then contract in volume, the sensing apparatus further includes a determination unit that determines whether or not a difference between an amplitude value of a pulse wave detected by the detector before or after a period of time during which the calibration device measures the first biological information, and an amplitude value of the pulse wave detected by the detector when the cuff expands to the maximum during the period of time is equal to or greater than a third threshold value, and the wear-determination unit makes a notification of incorrect manner of wearing if the difference is determined as being smaller than the third threshold value.

According to a sixth aspect of the present invention, a biological information measuring apparatus includes a sensing apparatus and a calibration device, the calibration device including: a measuring device that intermittently measures first biological information; a transmitter that transmits data including the first biological information; the sensing apparatus including: a receiver that receives the data; a detector that detects a pulse wave continuously in time; a determination unit that determines whether or not a difference between an amplitude value of a pulse wave detected by the detector before or after a period of time during which the calibration device measures the first biological information, and a minimum amplitude value of the pulse wave detected by the detector during the period of time is equal to or greater than a threshold value; and a calculator that calibrates the pulse wave based on the first biological information and calculates second biological information based on the calibrated pulse wave if it is determined that the difference is equal to or greater than the threshold value.

According to a seventh aspect of the present invention, a biological information measuring apparatus includes a sensing apparatus and a calibration device, the calibration device including: a measuring device that intermittently measures first biological information; a cuff that is being worn on a living body when the measuring device measures the first biological information, and configured to expand and then contract in volume; and a transmitter that transmits data including the first biological information and information on expansion of the cuff, and the sensing apparatus including: a receiver that receives the data; a detector that detects a pulse wave continuously in time; a determination unit that determines whether or not a difference between an amplitude value of a pulse wave detected by the detector before or after a period of time during which the calibration device measures the first biological information, and an amplitude value of the pulse wave detected by the detector when the cuff expands to the maximum during the period of time is equal to or greater than a third threshold value; and a calculator that calibrates the pulse wave based on the first biological information and calculates second biological information based on the calibrated pulse wave if it is determined that the difference is equal to or greater than the third threshold value.

According to an eighth aspect of the present invention, the measuring device measures the first biological information with higher precision than second biological information obtained from the detector.

According to a ninth aspect of the present invention, the detector detects the pulse wave beat by beat, and the first biological information and the second biological information are blood pressures.

According to the first aspect of the present invention, the sensing apparatus includes: the receiver that receives the first biological information and the calibration position information; and the detector that detects the pulse wave continuously in time and the sensing apparatus is separated from the calibration device. Thereby, the sensing apparatus is made compact, allowing the sensor to be disposed at a position where the pulse wave can be acquired more reliably. The calibration device includes: the measuring device that intermittently measures first biological information; the calibration position calculator that calculates calibration position information on the position of the calibration device; and the transmitter that transmits the calibration position information and data including the first biological information to the sensing apparatus. It is thereby possible for the sensing apparatus to calculate biological information based on the pulse wave with high precision, allowing the user to easily obtain high-precision biological information. In addition, since the measuring device performs measurement only intermittently, the period of time during which the measuring device interferes with the user is reduced. Moreover, since the calibration device is independently provided, the calibration device can be mounted at a position appropriate for calibration with ease, regardless of the disposition of the sensing apparatus. Furthermore, the sensor position information on the position of the sensing apparatus is calculated, and determination is made as to whether or not the sensing apparatus and the calibration device are being worn on the identical biological site based on the sensor position information and the calibration position information. It is thereby possible to know whether or not the sensing apparatus and the calibration device are being correctly worn, and to know whether or not the second biological information calculated by the calibration device based on the pulse wave is reliable.

According to the second aspect of the present invention, at least one of the acceleration sensor and the atmospheric pressure sensor is provided in each of the sensing apparatus and the calibration device, and the acceleration and the atmospheric-pressure-based height can be measured with respect to each of the sensing apparatus and the calibration device. It is thereby possible to determine whether or not the sensing apparatus and the calibration device are being worn on the same biological site.

According to the third aspect of the present invention, it can be seen that the sensing apparatus and the calibration device make substantially the same movement if the difference in acceleration of the acceleration sensor between the sensor position calculator and the calibration position calculator during a certain period of time is equal to or below the first threshold value. Moreover, it can be seen that the sensing apparatus and the calibration device are at substantially the same height if the difference in atmospheric pressure of the atmospheric pressure sensor between the sensor position calculator and the calibration position calculator during the certain period of time is equal to or below the second threshold value. If the changes in the above-described movement and height are substantially the same between the sensing apparatus and the calibration device, it can be regarded that the sensing apparatus and the calibration device are being worn on the same biological site.

According to the fourth aspect of the present invention, determination is made as to whether or not the difference between the amplitude value of the pulse wave detected by the detector before or after the period of time during which the calibration device measures the first biological information, and the minimum amplitude value of the pulse wave detected by the detector during the period of time is equal to or greater than the third threshold value, and the sensing apparatus is disposed at a position separated from the heart (e.g., on the palm side), as compared to the calibration device; if the sensing apparatus and the calibration device are being worn at the normal, correct position, and the amplitude value of the pulse wave decreases when the blood pressure measurement is performed at the calibration device, it is possible to determine whether or not the positional relationship (e.g., the positional relationship in the direction in which the arm extends) between the sensing apparatus and the calibration device based on the distance from the heart is correct. In addition, a notification of incorrect manner of wearing is made if the difference is determined as being smaller than the third threshold value. This allows the user to wear the sensing apparatus and the calibration device correctly.

According to the fifth aspect of the present invention, the cuff that is being worn on the living body during measurement of the first biological information by the measuring device, and configured to expand and then contract in volume is provided, and determination is made as to whether or not the difference between the amplitude value of the pulse wave detected by the detector before or after the period of time during which the calibration device measures the first biological information, and the amplitude value of the pulse wave detected when the cuff expands to the maximum during the period of time is equal to or greater than the third threshold value. It is thereby possible to determine whether or not the pulse wave has attenuated when the sensing apparatus is being worn at a position separated from the heart, as compared to the calibration device. Consequently, it is possible to determine the positional relationship between the sensing apparatus and the calibration device based on the distance from the heart. In addition, a notification of incorrect manner of wearing is made if the difference is determined as being smaller than the third threshold value. This allows the user to wear the sensing apparatus and the calibration device correctly.

According to the sixth aspect of the present invention, the sensing apparatus includes: the receiver that receives the data including the first biological information; the detector that detects the pulse wave continuously in time; the determination unit that determines whether or not the difference between the amplitude value of the pulse wave detected by the detector before or after the period of time during which the calibration device measures the first biological information, and the minimum amplitude value of the pulse wave detected by the detector during the period of time is equal to or greater than the threshold value; and the calculator that calibrates the pulse wave based on the first biological information and calculates second biological information based on the calibrated pulse wave if it is determined that the difference is equal to or greater than the threshold value, and the sensing apparatus is separated from the calibration device. Thereby, the sensing apparatus is made compact, and the sensor can be disposed at a position where the pulse wave can be acquired more reliably. The calibration device includes: the measuring device that intermittently measures first biological information; and the transmitter that transmits data including the first biological information. It is thereby possible for the sensing apparatus to calculate biological information with high precision based on the pulse wave, allowing the user to easily obtain high-precision biological information. In addition, since the measuring device performs measurement only intermittently, the period of time during which the measuring device interferes with the user is reduced. Moreover, since the calibration device is independently provided, the calibration device can be mounted at a position appropriate for calibration with ease, regardless of the disposition of the sensing apparatus. Furthermore, determination is made as to whether or not the difference between the amplitude value of the pulse wave detected by the detector before or after the period of time during which the calibration device measures the first biological information, and the minimum amplitude value of the pulse wave detected by the detector during the period of time is equal to or greater than the threshold value. Thereby, when the sensing apparatus and the calibration device are being worn at the normal, correct position, the sensing apparatus is disposed at a position separated from the heart (e.g., on the palm side), as compared to the calibration device, and the amplitude value of the pulse wave decreases when the blood pressure measurement is performed at the calibration device, it is possible to determine whether or not the positional relationship (e.g., the positional relationship in the direction in which the arm extends) between the sensing apparatus and the calibration device, based on the distance from the heart, is correct.

According to the seventh aspect of the present invention, the sensing apparatus includes: a receiver that receives the data including the first biological information; a detector that detects a pulse wave continuously in time; a determination unit that determines whether or not a difference between an amplitude value of a pulse wave detected by the detector before or after a period of time during which the calibration device measures the first biological information, and an amplitude value of a pulse wave detected by the detector when the cuff expands to the maximum during the period of time is equal to or greater than a third threshold value; and a calculator that calibrates the pulse wave based on the first biological information and calculates second biological information from the calibrated pulse wave if it is determined that the difference is equal to or greater than the third threshold value, and the sensing apparatus is separated from the calibration device. Thereby, the sensing apparatus is made compact, and the sensor can be disposed at a position where the pulse wave can be acquired more reliably. The calibration device includes: the measuring device that intermittently measures the first biological information; the cuff that is being worn on the living body when the measuring device measures the first biological information, and configured to expand and then contract in volume; and the transmitter that transmits data including the first biological information and information on expansion of the cuff, and the sensing apparatus calibrates the pulse wave based on the first biological information, calculates the second biological information from the calibrated pulse wave, and calibrates the pulse wave based on biological information measured by the measurement unit. It is thereby possible to calculate biological information with higher precision from the pulse wave, allowing the user to easily obtain high-precision biological information. In addition, since the measuring device performs measurement only intermittently, the period of time during which the measuring device interferes with the user is reduced. Moreover, since the calibration device is independently provided, the calibration device can be mounted at a position appropriate for calibration with ease, regardless of the disposition of the sensing apparatus. Furthermore, the cuff that is being worn on a living body when the measuring device measures the first biological information, and configured to expand and then contract in volume is provided, and determination is made as to whether or not a difference between an amplitude value of a pulse wave detected by the detector before or after a period of time during which the calibration device measures the first biological information, and an amplitude value of a pulse wave detected by the detector when the cuff expands to the maximum during the period of time is equal to or greater than a third threshold value. It is thereby possible to determine whether or not the pulse wave has attenuated by the cuff when the sensing apparatus is being worn at a position separated from the heart, as compared to the calibration device. Consequently, it is possible to determine the positional relationship between the sensing apparatus and the calibration device based on the distance from the heart.

According to the eighth aspect of the present invention, the first biological information is measured with higher precision than the second biological information obtained from the detector, and high-precision biological information is obtained from the measuring device for calibration. This ensures the precision of the biological information obtained based on the pulse wave from the detector, enabling calculation of the biological information with high precision continuously in time.

According to the ninth aspect of the present invention, the detection unit detects the pulse wave beat by beat, and the first biological information and the second biological information are blood pressures. It is thereby possible for the biological information measuring apparatus to measure the blood pressure of each beat of the pulse wave continuously in time.

That is, according to each aspect of the present invention, it is possible to provide a biological information measuring apparatus which, through being worn constantly, is capable of calibrating biological information continuously in time and acquiring accurate information, and a method and a program using the same.

### BRIEF DESCRIPTION OF THE DRAWINGS

FIG. 1 is a block diagram illustrating a blood pressure measuring apparatus according to a first embodiment.

FIG. 2 is a diagram illustrating an example in which the blood pressure measuring apparatus of FIG. 1 is being worn on the wrist.

FIG. 3 is a diagram illustrating another example in which the blood pressure measuring apparatus of FIG. 1 is being worn on the wrist.

FIG. 4 is a diagram illustrating the time course of the cuff pressure and the pulse wave signal by oscillometric technique.

FIG. 5 is a diagram illustrating beat-by-beat changes in pulse pressure over time, and a pulse wave of one of the heartbeats.

FIG. 6 is a flowchart illustrating a first calibration technique.

FIG. 7 is a flowchart for determining whether or not the sensing apparatus 110 and the calibration device 150 of FIG. 1 are being worn on the same arm.

FIG. 8 is a block diagram illustrating a blood pressure measuring apparatus according to a second embodiment.

FIG. 9 is a flowchart for determining whether or not the sensing apparatus and the calibration device of the blood pressure measuring apparatus of FIG. 8 are correctly disposed.

### DETAILED DESCRIPTION

Hereinafter, a biological information measuring apparatus and a method and a program using the same according to embodiments of the present invention will be described, with reference to the accompanying drawings. In the embodiments described below, components assigned with the same reference numbers are assumed to perform similar operations, and redundant descriptions thereof will be omitted.

The present embodiments have been made in response to the above-described circumstances, and aim to provide a biological information measuring apparatus which, through being worn constantly, is capable of acquiring accurate information while calibrating biological information continuously in time, and a method and a program using the same.

### First Embodiment

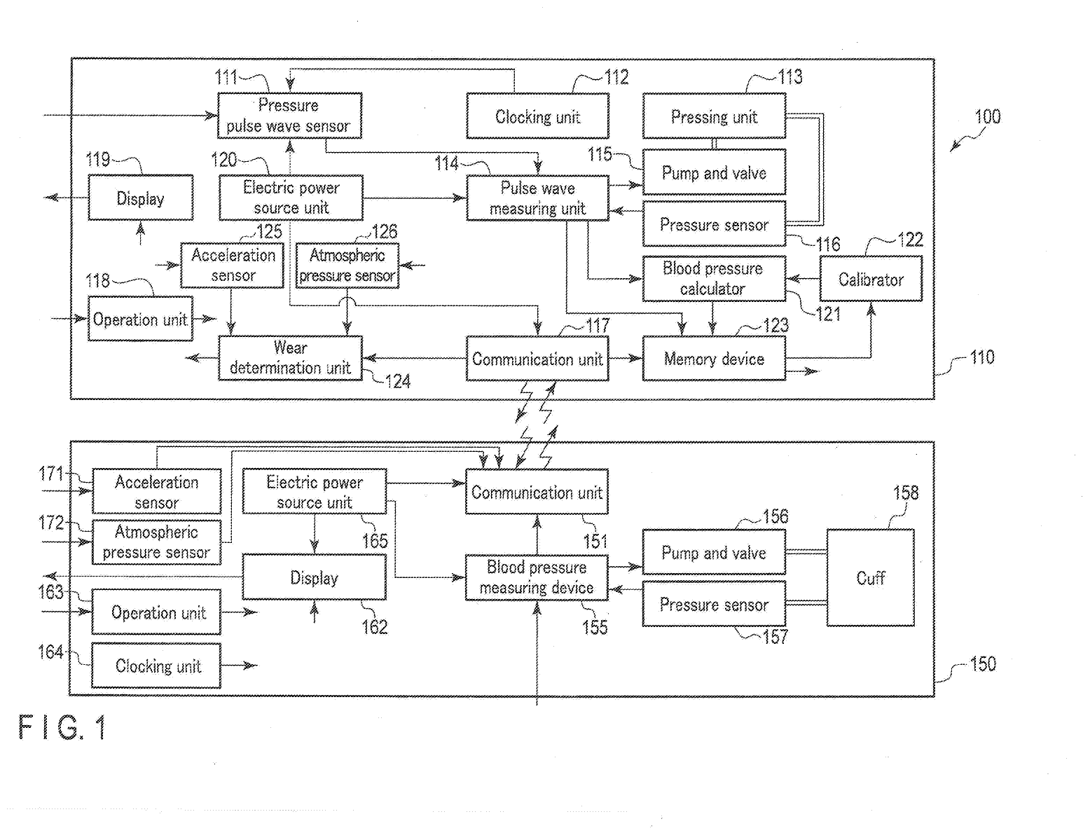

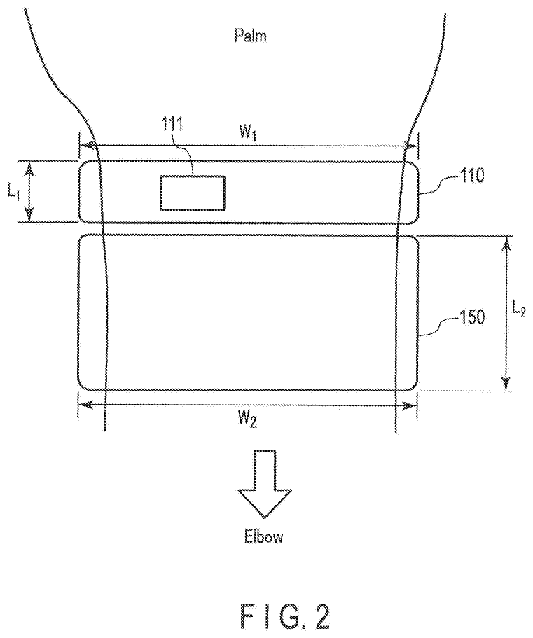

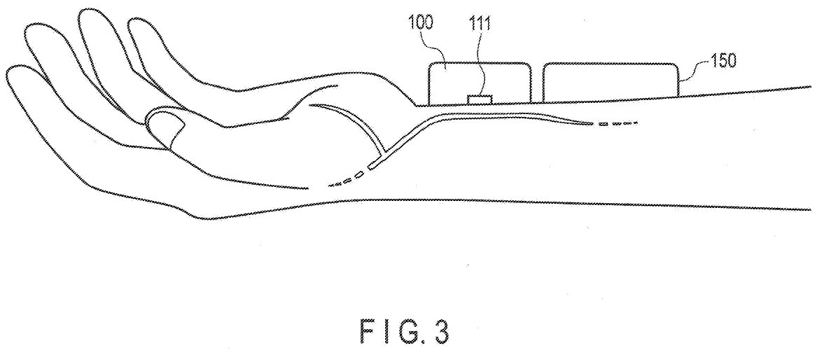

A blood pressure measuring apparatus 100 according to the present embodiment will be described, with reference to FIGS. 1, 2 and 3. FIG. 1 is a functional block diagram of the blood pressure measuring apparatus 100, illustrating details of a sensing apparatus 110 and a calibration device 150. FIG. 2 is a schematic perspective view, illustrating an example in which the blood pressure measuring apparatus 100 is being worn on the wrist, as seen from above the palm. A pressure pulse wave sensor 111 is disposed on the wrist side of the sensing apparatus 110. FIG. 3 is a schematic perspective view conceptually illustrating the blood pressure measuring apparatus 100 when being worn, as seen from the lateral side of the palm (i.e., the direction in which the fingers are aligned when the hand is open). FIG. 3 illustrates an example in which the pressure pulse wave sensor 111 is disposed orthogonal to the radial artery. It may appear from FIG. 3 that the blood pressure measuring apparatus 100 is simply laid on the palm side of the arm; however, the blood pressure measuring apparatus 100 is actually wrapped around the arm.

The blood pressure measuring apparatus 100 includes the sensing apparatus 110 and the calibration device 150. The sensing apparatus 110 includes the pressure pulse wave sensor 111, a clocking unit 112, a pressing unit 113, a pulse wave measuring unit 114, a pump and valve 115, a pressure sensor 116, a communication unit 117, an operation unit 118, a display 119, an electric power source unit 120, an acceleration sensor 125, an atmospheric pressure sensor 126, a blood pressure calculator 121, a calibrator 122, a memory device 123, and a wear-determination unit 124. The calibration device 150 includes a communication unit 151, a blood pressure measuring device 155, a pump and valve 156, a pressure sensor 157, a cuff 158, a display 162, an operation unit 163, a clocking unit 164, an acceleration sensor 171, and an atmospheric pressure sensor 172.

The blood pressure measuring apparatus 100 is circular, wrapped around the wrist, etc. like a bracelet, and measures the blood pressure based on biological information. The sensing apparatus 110 is disposed on a side of the wrist closer to the palm than the calibration device 150, as shown in FIGS. 2 and 3. In other words, the sensing apparatus 110 is disposed farther from the elbow than the calibration device 150. In the present embodiment, the sensing apparatus 110 is disposed in such a manner that the pressure pulse wave sensor 111 is positioned above the radial artery, and, in accordance with this disposition, the calibration device 150 is disposed on the side closer to the elbow than the sensing apparatus 110. The sensing apparatus 110 and the calibration device 150 may be worn on different arms. It is generally preferable that the sensing apparatus 110 and the calibration device 150 be disposed at the same height. It is further preferable to dispose the sensing apparatus 110 and the calibration device 150 at the height of the heart.

A length L₁ of the sensing apparatus 110 is set to be smaller than a length L₂ of the calibration device 150, as seen in the direction in which the arm extends. The length L₁ of the sensing apparatus 110 in the direction in which the arm extends is set to 40 mm or less, and more desirably, to 15 to 25 mm. A width W₁ of the sensing apparatus 110 is set to 4 to 5 cm, and a width W₂ of the calibration device 150 is set to 6 to 7 cm, as seen in the direction perpendicular to the direction in which the arm extends. In addition, the width W₁ and the width W₂ satisfy the relationship expressed as: 0 (or 0.5) cm<W₂−W₁<2 cm. Such a relationship prevents W₂ from being set too great, suppressing interference with the surroundings. By setting the size of the sensing apparatus 110 within such a range, the calibration device 150 is disposed on the side closer to the palm, thus facilitating detection of the pulse wave and keeping the precision in measurement. However, the calibration device 150 may be disposed on the upper arm during measurement.

The pressure pulse wave sensor 111 detects the pressure pulse wave continuously in time. For example, the pressure pulse wave sensor 111 detects the pressure pulse wave beat by beat. The pressure pulse wave sensor 111 is disposed on the palm side, as shown in FIG. 2, and is usually disposed parallel to the direction in which the arm extends, as shown in FIG. 3. The pressure pulse wave sensor 111 is capable of obtaining time-series data of the blood pressure value (blood pressure waveform), which changes according to the heartbeat.

The clocking unit 112 outputs time-of-day data to the pressure pulse wave sensor 111. The clocking unit 112 allows the pressure pulse wave sensor 111 to pass data on the pressure pulse wave, as well as the time-of-day data, to another component.

The pressing unit 113 is an air bag that presses the sensor portion of the pressure pulse wave sensor 111 against the wrist, thereby increasing the sensitivity of the sensor.

The pulse wave measuring unit 114 receives the pressure pulse wave data, as well as the time-of-day data, from the pressure pulse wave sensor 111, and passes the received data to the memory device 123 and the blood pressure calculator 121. The pulse wave measuring unit 114 controls the pump and valve 115, and the pressure sensor 116, to pressurize or depressurize the pressing unit 113, and adjusts the pressure pulse wave sensor 111 so as to be pressed against the radial artery at the wrist.

The communication unit 117 and the communication unit 151 communicate with each other by a communication system that enables short-distance data exchange. These communication units use, for example, a short-distance wireless communication system, such as Bluetooth (registered trademark), TransferJet (registered trademark), ZigBee (registered trademark), and IrDA (registered trademark).

The pump and valve 115 either pressurizes or depressurizes the pressing unit 113 according to an instruction from the pulse wave measuring unit 114. The pressure sensor 116 monitors the pressure of the pressing unit 113, and notifies the pulse wave measuring unit 114 of the pressure value of the pressing unit 113.

The electric power source unit 120 supplies electric power to each component of the sensing apparatus 110.

The acceleration sensor 125 measures the acceleration of the sensing apparatus 110, and outputs time-of-day data and the acceleration at that time of day. The acceleration sensor 125 measures, for example, accelerations with respect to the three spatial axes x, y and z, and obtains time-series acceleration data.

The atmospheric pressure sensor 126 measures the atmospheric pressure at the position of the sensing apparatus 110. The atmospheric pressure sensor 126 further calculates the altitude of the sensing apparatus based on the atmospheric pressure at the position of the sensing apparatus 110.

The blood pressure measuring device 155 measures the blood pressure, which is biological information, with higher precision than the pressure pulse wave sensor 111. The blood pressure measuring device 155 measures, for example, the blood pressure intermittently, not continuously in time, and passes the measured values to the memory device 123 and the calibrator 122 via the communication units 151 and 117. The blood pressure measuring device 155 measures the blood pressure using, for example, oscillometric technique. Moreover, the blood pressure measuring device 155 controls the pump and valve 156 and the pressure sensor 157 to pressurize or depressurize the cuff 158, thereby measuring the blood pressure. The blood pressure measuring device 155 passes data on the systolic blood pressure, as well as the time of day of measurement thereof, and data on the diastolic blood pressure, as well as the time of day of measurement thereof, to the memory device 123, via the communication units 151 and 117. The systolic blood pressure is also referred to as “SBP”, and the diastolic blood pressure is also referred to as “DBP”.

The memory device 123 sequentially acquires and stores data on the pressure pulse wave from the pulse wave measuring unit 114, as well as the time of day of detection thereof, and acquires and stores data on the SBP and the DBP from the blood pressure measuring device 155, as well as the times of day of measurement thereof, at the time of operation of the blood pressure measuring device 155, via the communication units 151 and 117. Also, the memory device 123 records, in association with the measured biological information, model information and/or unique identification information of the calibration device, in which first biological information for calibration is measured (by the blood pressure measuring device 155), used to calculate the measured biological information (continuous blood pressures). It is thereby possible to know, from the measured biological information, which blood pressure monitor (model, unique device number, etc.) has been used for the calibration.

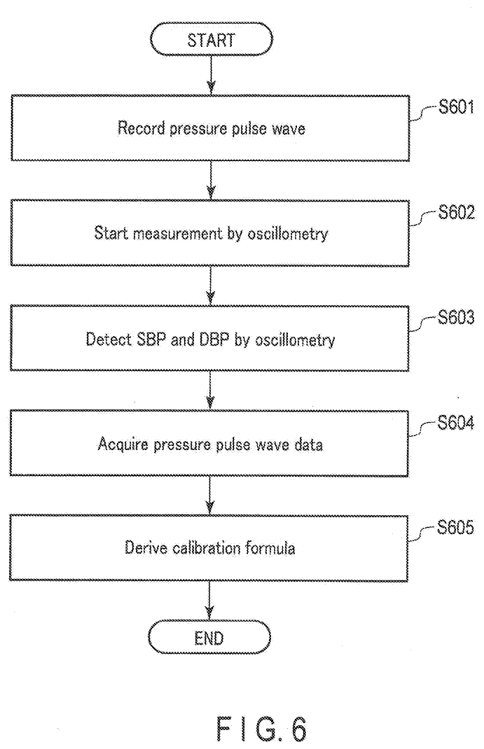

The calibrator 122 acquires, from the memory device 123, the data on the SBP and DBP, measured by the blood pressure measuring device 155 as well as the time of day of measurement thereof, and the data on the pressure pulse wave measured by the pulse wave measuring unit 114 of the sensing apparatus 110, as well as the time of day of measurement thereof. The calibrator 122 calibrates the pressure pulse wave from the pulse wave measuring unit 114, based on the blood pressure value from the blood pressure measuring device 155. Of several calibration techniques that may be adopted by the calibrator 122, an example calibration technique will be described later in detail, with reference to FIG. 6.

The blood pressure calculator 121 receives a calibration technique from the calibrator 122, calibrates the pressure pulse wave data from the pulse wave measuring unit 114, and stores the blood pressure data obtained from the pressure pulse wave data in the memory device 123, as well as the time-of-day-of-measurement data.

An electric power source unit 165 supplies electric power to each component of the calibration device 150.

The display 162 displays various types of information, such as the result of the blood pressure measurement, to the user. The display 162 receives data from, for example, the blood pressure measuring device 155, and displays the contents of the received data. For example, the display 162 displays the blood pressure value data, as well as the time-of-day-of-measurement data.

The display 119 also displays various types of information, such as the result of the blood pressure measurement, to the user. The display 119 receives data from, for example, the pulse wave measuring unit 114, and displays the contents of the received data. For example, the display 119 displays the pressure pulse wave data, as well as the time-of-day-of-measurement data.

The operation unit 163 receives an operation from the user. The operation unit 163 includes, for example, an operation button for causing the blood pressure measuring device 155 to start measurement, an operation button for performing calibration, and an operation button for initiating or terminating communication.

The operation unit 118 also receives an operation from the user. The operation unit 118 includes, for example, an operation button for triggering the pulse wave measuring unit 114 to start measurement, and an operation button for initiating or terminating communication.

The clocking unit 164 generates time-of-day data and supplies the generated time-of-day data to a component that requires such data. The memory device 123 records, for example, the time-of-day data, as well as data to be stored therein.

The acceleration sensor 171 measures the acceleration of the calibration device 150, and outputs the time-of-day data and the acceleration at that time of day (i.e., outputs time-series acceleration data). Similarly to the acceleration sensor 125, the acceleration sensor 171 measures accelerations with respect to the three axes, and obtains time-series acceleration data.

The atmospheric pressure sensor 172 measures the atmospheric pressure at the position of the calibration device 150. The atmospheric pressure sensor 172 further calculates the altitude of the calibration device 150 from the atmospheric pressure at the position of the calibration device 150.

The wear-determination unit 124 acquires the time-series acceleration data from the acceleration sensor 125 and the time-series atmospheric pressure data from the atmospheric pressure sensor 126 of the sensing apparatus 110, and further acquires the time-series acceleration data and the time-series atmospheric pressure data from the acceleration sensor 171 and the atmospheric pressure sensor 172, via the communication units 117 and 151. Based on the acquired acceleration data and atmospheric pressure data, the wear-determination unit 124 determines whether or not the sensing apparatus 110 and the calibration device 150 are being worn on the same biological site. The wear-determination unit 124 determines, for example, whether or not the sensing apparatus 110 and the calibration device 150 are being worn on the same arm. The wear-determination unit 124 estimates the positional relationship between the sensing apparatus 110 and the calibration device 150 to determine whether or not the sensing apparatus 110 and the calibration device 150 are being correctly worn.

At the time of implementation, a program for executing each of the above-described operations is stored in, for example, a secondary storage device included in each of the pulse wave measuring unit 114, the calibrator 122, the blood pressure calculator 121, and the blood pressure measuring device 155, and the central processing unit (CPU) executes a read operation of the stored program. The secondary storage device is, for example, a hard disk; however, it may be any device capable of storing data, such as a semiconductor memory, a magnetic memory device, an optical memory device, a magneto-optical disk, and a memory device employing the phase-change recording technology.

In addition, a program for executing an operation to be performed by each of the pulse wave measuring unit 114, the calibrator 122, the blood pressure calculator 121, and the blood pressure measuring device 155 may be stored in a server, etc., separate from the sensing apparatus and the calibration device, and the stored program may be executed therein. In this case, pulse wave data measured by the sensing apparatus and blood pressure data measured by the calibration device as biological information may be transmitted to and calibrated by the server, thus allowing the server to obtain the blood pressure based on the pulse wave. Since the processing is performed by the server in such a case, the processing speed may increase. Moreover, since the pulse wave measuring unit 114, the calibrator 122, the blood pressure calculator 121, and the blood pressure measuring device 155 are reduced in size, with the respective device parts removed from the sensing apparatus and the calibration device, the sensor can be easily disposed at a position where accurate measurement can be performed. This reduces the burden on the user, leading to a simple and accurate blood pressure measurement.

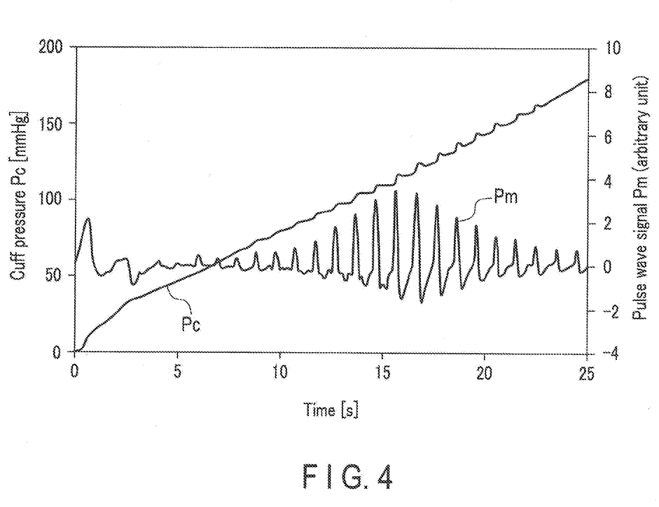

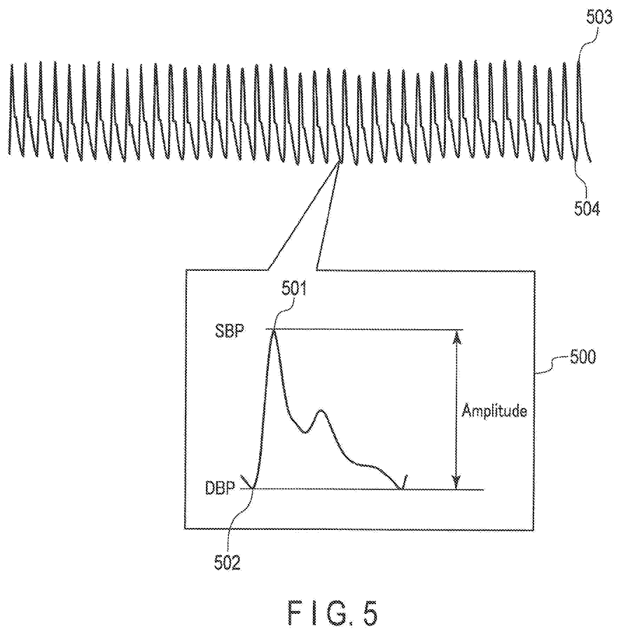

Next, operations performed by the pulse wave measuring unit 114 and the blood pressure measuring device 155 prior to calibration by the calibrator 122 will be described, with reference to FIGS. 4 and 5. FIG. 4 illustrates changes in cuff pressure and changes in magnitude of the pulse wave signal over time, during a blood pressure measurement by oscillometric technique. It can be seen from FIG. 4, illustrating changes in cuff pressure and changes in pulse wave signal over time, that the cuff pressure increases with time, and that the magnitude of the pulse wave signal gradually increases with the increase in the cuff pressure, and gradually decreases after reaching the maximum value. FIG. 5 illustrates time-series pulse pressure data acquired by beat-by-beat measurement of the pulse pressure. FIG. 5 also illustrates a waveform of a pressure pulse wave of one of the pulses.

A brief description will be given of the operation when the blood pressure measuring device 155 performs a blood pressure measurement by oscillometric technique, with reference to FIG. 4. The blood pressure value may be calculated not only in the course of pressurization, but also in the course of depressurization; however, only the course of pressurization is illustrated as an example.

When the user instructs a blood pressure measurement by oscillometric technique via the operation unit 163 provided in the calibration device 150, the blood pressure measuring device 155 commences operation, and initializes the memory area for processing. Moreover, the blood pressure measuring device 155 deactivates the pump of the pump and valve 156 to open the valve, and allows the air in the cuff 158 to be discharged. Subsequently, control is performed to set the output value of the pressure sensor 157 at that point in time as a value corresponding to the atmospheric pressure (adjusted to 0 mmHg).

Subsequently, the blood pressure measuring device 155 functions as a pressure controller and performs control to deliver air to the cuff 158 by closing the valve of the pump and valve 156 and then driving the pump. This expands the cuff 158, and gradually increases the cuff pressure (Pc in FIG. 4) for pressurization. To calculate blood pressure values in the course of pressurization, the blood pressure measuring device 155 monitors the cuff pressure Pc using the pressure sensor 157, and acquires, as the pulse wave signal Pm as shown in FIG. 4, the fluctuation component of the arterial volume generated in the radial artery of the wrist, which is the measurement site.

Thereafter, the blood pressure measuring device 155 attempts to calculate the blood pressure values (SBP and DBP) based on the pulse wave signal Pm acquired at that point in time, by applying a known algorithm using oscillometric technique. If the blood pressure values cannot be calculated yet at this point in time because of shortage of data, a pressurization treatment similar to the above-described one is repeated, unless the cuff pressure Pc reaches the upper-limit pressure (which is preset to, for example, 300 mmHg for safety).

After the blood pressure values are thus calculated, the blood pressure measuring device 155 performs control to discharge the air in the cuff 158, by deactivating the pump of the pump and valve 156 in order to open the valve. Lastly, the results of measurement of the blood pressure values are passed to the calibration unit.

Next, a description will be given of the beat-by-beat measurement of the pulse wave by the pulse wave measuring unit 114, with reference to FIG. 5. The pulse wave measuring unit 114 measures the pulse wave using, for example, tonometry.

In order for the pressure pulse wave sensor 111 to realize the optimum measurement, the pulse wave measuring unit 114 controls the pump and valve 115 and the pressure sensor 116 to reach a predetermined optimum pressing force, by increasing the internal pressure of the pressing unit 113 to the optimum pressing force and maintaining the optimum pressing force. Next, when the pressure pulse wave is detected by the pressure pulse wave sensor 111, the pulse wave measuring unit 114 acquires the detected pressure pulse wave.

The pressure pulse wave is continuously detected beat by beat as a waveform as shown in FIG. 5. The pressure pulse wave 500 in FIG. 5 represents a pressure pulse wave of one beat, with a pressure value 501 corresponding to the SBP and a pressure value 502 corresponding to the DBP. Normally, the SBP 503 and the DBP 504 fluctuate according to the heartbeat of the pressure pulse, as shown by the time-series pressure pulse wave in FIG. 5.

Next, a description will be given of the operation of the calibrator 122, with reference to FIG. 6.

The calibrator 122 calibrates the pressure pulse wave detected by the pulse wave measuring unit 114, using the blood pressure value measured by the blood pressure measuring device 155. That is, the calibrator 122 determines the maximum value 501 and the minimum value 502 of the blood pressure values of the pressure pulse wave detected by the pulse wave measuring unit 114.

(Calibration Techniques)

The pulse wave measuring unit 114 starts recording data on the pressure pulse wave, as well as the time of day of measurement thereof, and sequentially stores the pressure pulse wave data in the memory device 123 (step S601). Thereafter, measurement by oscillometric technique is started by, for example, a user who activates the blood pressure measuring device 155 using the operation unit 163 (step S602). The blood pressure measuring device 155 records SBP data and DBP data, as well as the times of day of detection of the SBP and DBP by oscillometric technique, based on the pulse wave signal Pm, and stores the recorded SBP data and DBP data in the memory device 123 (step S603). The calibrator 122 acquires a pressure pulse wave corresponding to the SBP data and the DBP data from the pressure pulse wave data (step S604). The calibrator 122 derives a calibration formula based on the maximum value 501 of the pressure pulse wave corresponding to the SBP and the minimum value 502 of the pressure pulse wave corresponding to the DBP (step S605).

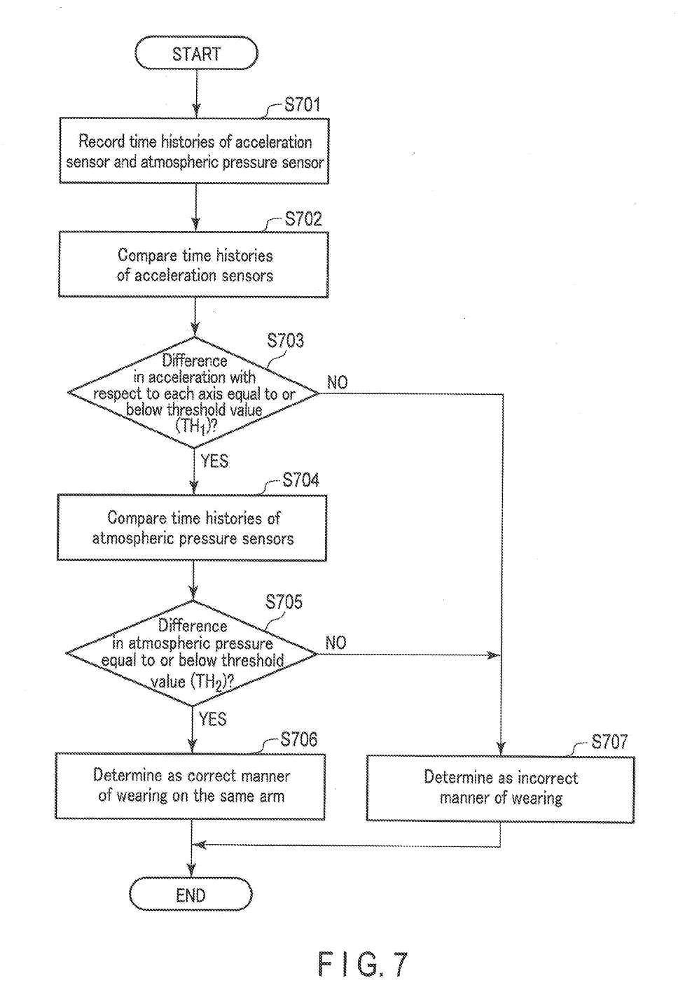

Next, a description will be given, with reference to FIG. 7, of the determination by the wear-determination unit 124 as to whether or not the sensing apparatus 110 and the calibration device 150 are being worn on the same arm, based on analysis of data from the acceleration sensor and the atmospheric pressure sensor integrated in each of the sensing apparatus 110 and the calibration device 150 of the present embodiment. The steps of FIG. 7 are executed by the wear-determination unit 124 of the sensing apparatus 110. However, such steps may be executed by the calibration device 150 including the wear-determination unit 124. The operations of the wear-determination unit 124 (to be described below) may be performed in such a manner that an external device, such as a server device, separated from the blood pressure measuring apparatus 100 receives required input information, performs calculation, and returns the calculated result to the blood pressure measuring apparatus 100.

The wear-determination unit 124 acquires and records time history information of the accelerations of the acceleration sensor 125 and time history information of the atmospheric pressures of the atmospheric pressure sensor 126 (step S701). The wear-determination unit 124 further acquires and records time history information of the accelerations from the acceleration sensor 171 and time history information of the atmospheric pressures from the atmospheric pressure sensor 172 via the communication units 117 and 151 (step S701).

The time history of the accelerations of the acceleration sensor 125 and the time history of the accelerations of the acceleration sensor 171 are compared (step S702). The wear-determination unit 124 designates a time range to be examined, and determines, for example, whether or not the difference between the acceleration of the acceleration sensor 125 and the acceleration of the acceleration sensor 171 with respect to each of the three axes is equal to or below a threshold value TH₁ within the designated time range (step S703). If the difference between the acceleration of the acceleration sensor 125 and the acceleration of the acceleration sensor 171 is equal to or below the threshold value TH₁ with respect to all the three axes, it is determined that the sensing apparatus 110 and the calibration device 150 are possibly being worn on the same arm, and the processing advances to step S704; if the difference in acceleration exceeds the threshold value with respect to any of the axes, the processing advances to step S707. The threshold value may be changed either according to the axis or according to the user in consideration of the user's behavior habit.

In step S704, the time history of the atmospheric pressure sensor 126 of the sensing apparatus 110 and the time history of the atmospheric pressure sensor 172 of the calibration device 150 are compared in a designated time range (step S704). In step S705, determination is made as to whether or not the difference between the atmospheric pressure sensor 126 of the sensing apparatus 110 and the atmospheric pressure sensor 172 of the calibration device 150 is equal to or below a threshold value TH₂ (step S705). If the difference between the atmospheric pressure sensor 126 and the atmospheric pressure sensor 172 is equal to or below the threshold value TH₂, the processing advances to step S706; if the difference between the atmospheric pressure sensor 126 and the atmospheric pressure sensor 172 exceeds the threshold value TH₂, the processing advances to step S707. In step S706, it is determined that the sensing apparatus 110 and the calibration device 150 are being worn on the same arm (step S706).

In step S707, it is determined that the sensing apparatus 110 and the calibration device 150 are not being worn on the same arm, and that either the sensing apparatus 110 or the calibration device 150 is being worn at an incorrect position. In step S707, the display 119 or the display 162 may display information that the sensing apparatus 110 and the calibration device 150 are not being worn the same arm, so as to warn the user. For example, the display 119 or the display 162 may display the message: “Should be worn on the same arm”.

According to the first embodiment described above, since the sensing apparatus 110 and the calibration device 150 are separated, the necessity to align the calibration device 150 is reduced, and the pressure pulse wave sensor 111 of the sensing apparatus 110 can be disposed at the optimum position. Since the pulse wave is calibrated based on the first blood pressure value measured by the calibration device 150, and the second blood pressure value is calculated based on the pulse wave, it is possible to calculate biological information with high precision based on the pulse wave, thus allowing the user to easily obtain high-precision biological information. Moreover, since the calibration device 150 is independently provided, the calibration device 150 can be mounted at a position appropriate for calibration with ease, regardless of the disposition of the sensing apparatus 110. By providing an acceleration sensor and an atmospheric pressure sensor in both the sensing apparatus 110 and the calibration device 150 and comparing their time histories, the histories of the movements and the heights of the sensing apparatus 110 and the calibration device 150 can be known, and the positional relationship between the sensing apparatus 110 and the calibration device 150 can be estimated. It is thereby possible to determine whether or not the sensing apparatus 110 and the calibration device 150 are being correctly worn.

### Second Embodiment

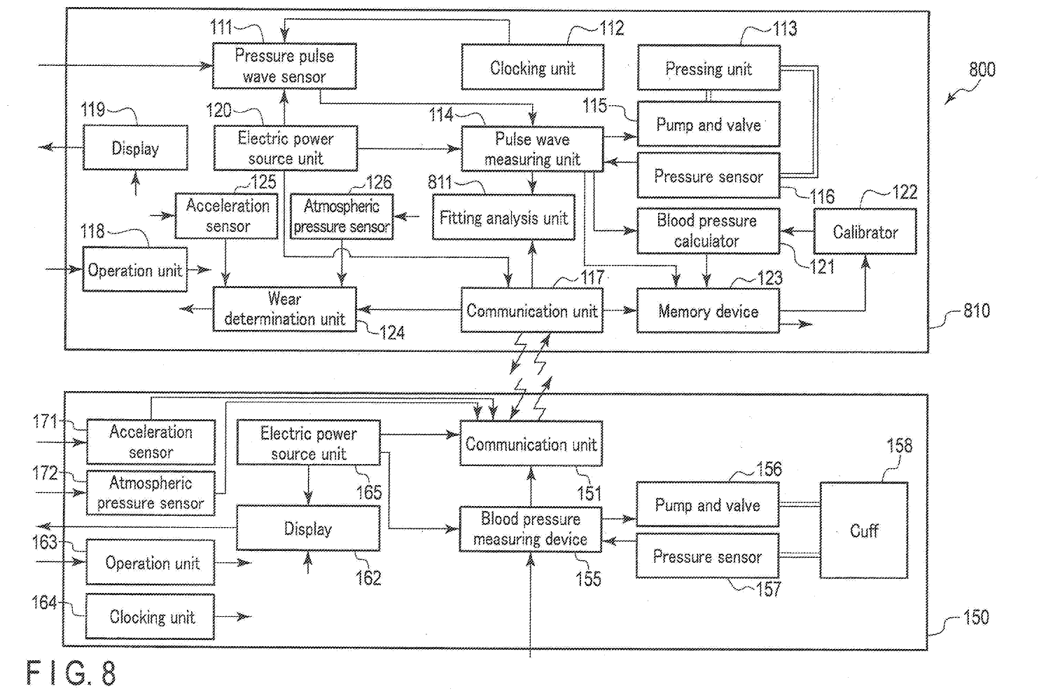

A blood pressure measuring apparatus 800 according to the present embodiment will be described, with reference to FIGS. 8, 2, and 3. FIG. 8 is a functional block diagram of the blood pressure measuring apparatus 800, illustrating details of the sensing apparatus 810 and the calibration device 150. The schematic perspective view of FIG. 2, illustrating an example in which the blood pressure measuring apparatus 100 is being worn on the wrist, as seen from above the palm, similarly applies to the blood pressure measuring apparatus 800. A pressure pulse wave sensor 111 is disposed on the wrist side of the sensing apparatus 110. The schematic perspective view of FIG. 3, conceptually illustrating the blood pressure measuring apparatus 100 when being worn, as seen from the lateral side of the palm (i.e., the direction in which the fingers are aligned when the hand is open), similarly applies to the blood pressure measuring apparatus 800. FIG. 3 illustrates an example in which the pressure pulse wave sensor 111 is disposed orthogonal to the radial artery. It may appear from FIG. 3 that the blood pressure measuring apparatus 100 is simply laid on the palm side of the arm; however, the blood pressure measuring apparatus 100 is actually wrapped around the arm. FIGS. 2 and 3 apply to the present embodiment, similarly to the first embodiment.

The blood pressure measuring apparatus 800 according to the present embodiment is the same as the blood pressure measuring apparatus 100 according to the first embodiment in terms of the calibration device, and differs therefrom only in terms of the sensing apparatus 810.

The sensing apparatus 810 of the present embodiment is the sensing apparatus 810 of the first embodiment, to which a fitting analysis unit 811 is added. The fitting analysis unit 811 acquires the pulse wave from the pulse wave measuring unit 114 and the blood pressure value from the blood pressure measuring device 155 via the communication units 117 and 151, monitors the time history of their waveforms before and after the operation of the blood pressure measuring device 155, and measures the fluctuation in amplitude of the pulse wave between the periods of time. Thereafter, the fitting analysis unit 811 determines whether or not the amplitude of the pulse wave is smaller than the threshold value, and determines, based on this determination, whether or not the sensing apparatus 110 and the calibration device 150 are disposed and being worn correctly. The fitting analysis unit 811 may display the result of determination on the displays 119 and 162.

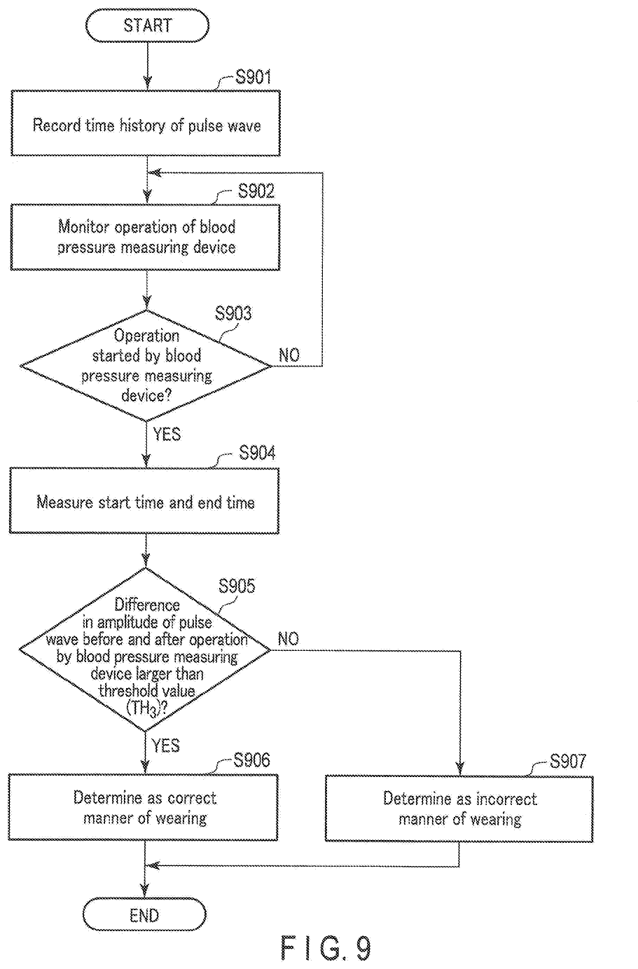

Next, an operation for determining whether or not the sensing apparatus 110 and the calibration device 150 are being correctly worn will be described, with reference to FIG. 9. FIG. 9 illustrates an operation performed by the fitting analysis unit 811 mounted in the sensing apparatus 810; however, the analysis unit may be provided in the calibration device 150, or may input required information to a server device provided outside the blood pressure measuring apparatus 800 and execute a program that executes the procedure shown in FIG. 9.

The fitting analysis unit 811 starts acquiring the time history of the pulse wave from the pulse wave measuring unit 114 of the sensing apparatus 110, and continues recording (step S901).

The fitting analysis unit 811 monitors the operation of the blood pressure measuring device 155, to determine whether or not the blood pressure measuring device 155 has started the measurement (step S902).

Upon determining whether or not the blood pressure measuring device 155 has started the measurement, if it is determined that the measurement has been started, the processing advances to step S904, and if it is determined that the measurement has not been started, the processing returns to step S902 (step S903).

In step S904, a start time when the blood pressure measuring device 155 has started measurement, and an end time when the measurement has been ended are measured. During this period of time, the time history of the pulse wave of the pulse wave measuring unit 114 is continuously recorded.

If the blood pressure measuring device 155 starts measurement and the cuff 158 starts to bulge, the amplitude value of the pulse wave of the pulse wave measuring unit 114 is acquired before and after the bulging of the cuff 158. Comparison is made between, for example, the amplitude value of the pulse wave of the pulse wave measuring unit 114 before the cuff 158 starts to bulge, and the amplitude value of the pulse wave measuring unit 114 when the cuff 158 starts to bulge and expands to the maximum. Upon comparing the difference between the amplitude values and determining whether or not the difference between the amplitude values is larger than the threshold value (TH₃), if the difference between the amplitude values is larger than the threshold value TH₃, the processing advances to step S906, and if the difference is not large, the processing advances to step S907 (step S905). Alternatively, the minimum amplitude value of the pulse wave of the pulse wave measuring unit 114, during the period from the start to the end of the measurement, by the blood pressure measuring device 155 using the cuff may be used instead of the amplitude value of the pulse wave of the pulse wave measuring unit 114 when the cuff 158 starts to bulge and expands to the maximum. The information on the expansion of the cuff 158 may be transmitted from the communication unit 151 to the communication unit 117, and determination may be made using the fitting analysis unit 811.

In step S906, corresponding to the case where the tightening of the cuff has a large influence on the pulse wave, the sensing apparatus 110 is regarded as being worn on the side closer to the palm than the calibration device 150, and it is determined that the blood pressure measuring apparatus 800 is being correctly worn. On the other hand, in step S907, corresponding to the case where the tightening of the cuff has little influence on the pulse wave, the sensing apparatus 110 is regarded as being on the side closer to the upper arm than the calibration device 150, and it is determined that the blood pressure measuring apparatus 800 is not being correctly worn.

Alternatively, the amplitude value of the pulse wave when the cuff expands to the maximum may be measured, as well as the blood pressure value by the blood pressure measuring device 155 at that time, and determination may be made as to whether or not the amplitude value of the pulse wave is smaller than a threshold value that is set using the blood pressure value (SBP or DBP) of the blood pressure measuring device 155 as a variable. In this case, the threshold value is set so as to increase as the blood pressure value measured by the blood pressure measuring device 155 increases, and if the amplitude value of the pulse wave is smaller than the set threshold value, it is determined that the blood pressure measuring apparatus 800 is not being correctly worn.

If the sensing apparatus 110 is regarded as being closer to the upper arm side than the calibration device 150, the user may be warned of the incorrect manner of wearing, and advised that the sensing apparatus 110 and the calibration device 150 be switched. For example, the display 119 or 162 may display information that recommends switching the sensing apparatus 110 and the calibration device 150 (e.g. “the sensing apparatus and the calibration device should be switched”).

According to the second embodiment described above, in addition to the effects of the first embodiment, it is possible to determine whether or not the relative positional relationship between the sensing apparatus 110 and the calibration device 150 is proper, by measuring the amplitude of the pulse wave detected by the pulse wave measuring unit 114, from when the blood pressure measuring device 155 starts measurement by increasing the cuff until the blood pressure measuring device 155 ends the measurement by decreasing the cuff, based on changes in the amplitude value of the pulse wave or the amplitude value of the pulse wave when the cuff is increased to the maximum.

In the above-described embodiment, the pressure pulse wave sensor 111 detects, for example, the pressure pulse wave of the radial artery passing through the measurement site (e.g., the left wrist) (method of tonometry). However, the configuration is not limited thereto. The pressure pulse wave sensor 111 may be configured to detect the pulse wave of the radial artery passing through a measurement site (e.g., the left wrist) as a change in impedance (impedance method). The pressure pulse wave sensor 111 may include a light-emitting element that emits light toward an artery passing through the corresponding portion of the measurement site, and a light-receiving element that receives reflected light (or transmitted light) of the emitted light, and may be configured to detect the pulse wave of the artery as changes in volume (photoelectric method). Moreover, the pressure pulse wave sensor 111 may include a piezoelectric sensor in contact with the measurement site, and may be configured to detect a strain caused by the pressure of the artery passing through the corresponding portion of the measurement site as a change in electric resistance (piezoelectric method). Furthermore, the pressure pulse wave sensor 111 may include a transmit element that transmits radio waves toward an artery passing through the corresponding portion of the measurement site, and a receive element that receives reflection waves of the transmitted radio waves, and may be configured to detect a change in distance between the artery and the sensor caused by the pulse wave of the artery as a phase shift between the transmission waves and the reflection waves (radio wave irradiation method). In addition to the above-described methods, any method that enables observation of a physical quantity based on which the blood pressure can be calculated may be adopted.

In the above-described embodiment, the blood pressure measuring apparatuses 100 and 800 are assumed to be worn on the left wrist, which is the measurement site; however, the configuration is not limited thereto, and they may be worn, for example, on the right wrist. The measurement site is not limited to the wrist and may be any part through which an artery passes; examples include an upper limb such as an upper arm and a lower limb such as an ankle and a thigh.

The apparatus of the present invention can also be realized by a computer and a program, and such a program may be recorded on a recording medium or provided through a network.

Moreover, the above-described apparatuses and their device portions can be implemented either as a hardware configuration or as a combined configuration of hardware resources and software. The software of the combined configuration may be a program pre-installed in a computer from a network or a computer-readable storage medium, to be executed by the processor of the computer in order to allow the computer to implement the functions of the respective apparatuses.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

Furthermore, part or all of the above-described embodiments may be described as in the additional descriptions given below; however, the embodiments are not limited thereto.

(Additional Description 1)

A biological information measuring apparatus comprising a sensing apparatus including a first hardware processor and a calibration device including a second hardware processor and a memory,

the second hardware processor being configured to:
intermittently measure first biological information;
calculate calibration position information on a position of the calibration device; and
transmit the calibration position information and data including the first biological information to the sensing apparatus,

the first hardware processor being configured to:
receive the first biological information and the calibration position information;
detect a pulse wave continuously in time;
calibrate the pulse wave based on the first biological information and calculate second biological information based on the calibrated pulse wave;
calculate sensor position information on a position of the sensing apparatus; and
determine whether or not the sensing apparatus and the calibration device are being worn on an identical biological site based on the sensor position information and the calibration position information, and

the memory including:
a memory device that stores the second biological information.

(Additional Description 2)

A biological information measuring apparatus comprising a sensing apparatus including a first hardware processor and a calibration device including a second hardware processor and a memory,

the second hardware processor being configured to:
intermittently measure first biological information; and
transmit data including the first biological information,

the first hardware processor being configured to:
receive the data;
detect a pulse wave continuously in time;
determine whether or not a difference between an amplitude value of a pulse wave detected before or after a period of time during which the calibration device measures the first biological information, and a minimum amplitude value of the pulse wave detected during the period of time is equal to or greater than a threshold value; and
calibrate the pulse wave based on the first biological information and calculate second biological information based on the calibrated pulse wave if it is determined that the difference is equal to or greater than the threshold value, and

the memory including:
a memory device that stores the second biological information.

(Additional Description 3)

A biological information measuring apparatus comprising a sensing apparatus including a first hardware processor and a calibration device including a second hardware processor and a memory,

the second hardware processor being configured to:
intermittently measure first biological information; and
transmit data including the first biological information and information on expansion of the cuff;

the first hardware processor being configured to:
receive the data;
detect a pulse wave continuously in time;
determine whether or not a difference between an amplitude value of a pulse wave detected before or after a period of time during which the calibration device measures the first biological information, and an amplitude value of the pulse wave detected when the cuff expands to the maximum during the period of time is equal to or greater than a third threshold value; and
calibrate the pulse wave based on the first biological information and calculate second biological information based on the calibrated pulse wave if it is determined that the difference is equal to or greater than the third threshold value, and

the memory including:
a memory device that stores the second biological information.

(Additional Description 4)

A method of measuring biological information, comprising:

intermittently measuring first biological information, using at least one hardware processor;

calculating calibration position information on a position of the calibration device, using the at least one hardware processor;

transmitting the calibration position information and data including the first biological information to the sensing apparatus, using the at least one hardware processor;

receiving the first biological information and the calibration position information, using the at least one hardware processor;

detecting a pulse wave continuously in time, using the at least one hardware processor;

calibrating the pulse wave based on the first biological information and calculating second biological information based on the calibrated pulse wave, using the at least one hardware processor;

calculating sensor position information on a position of the sensing apparatus, using the at least one hardware processor; and

determining whether or not the sensing apparatus and the calibration device are being worn on an identical biological site based on the sensor position information and the calibration position information, using the at least one hardware processor.

(Additional Description 5)

A method of measuring biological information, comprising:

intermittently measuring first biological information, using at least one hardware processor; and

transmitting data including the first biological information, using the at least one hardware processor; and

receiving the data using the at least one hardware processor;

detecting a pulse wave continuously in time, using the at least one hardware processor;

determining whether or not a difference between an amplitude value of a pulse wave detected before or after a period of time during which the calibration device measures the first biological information, and a minimum amplitude value of the pulse wave detected during the period of time is equal to or greater than a threshold value, using the at least one hardware processor; and

calibrating the pulse wave based on the first biological information and calculating second biological information based on the calibrated pulse wave if it is determined that the difference is equal to or greater than the threshold value, using the at least one hardware processor.

(Additional Description 6)

A method of measuring biological information, comprising:

intermittently measuring first biological information, using at least one hardware processor;

transmitting data including the first biological information and information on expansion of a cuff, using the at least one hardware processor;

receiving the data using the at least one hardware processor;

detecting a pulse wave continuously in time, using the at least one hardware processor;

determining whether or not a difference between an amplitude value of a pulse wave detected before or after a period of time during which the calibration device measures the first biological information, and an amplitude value of the pulse wave detected when the cuff expands to the maximum during the period of time is equal to or greater than a third threshold value, using the at least one hardware processor; and

calibrating the pulse wave based on the first biological information and calculating second biological information based on the calibrated pulse wave if it is determined that the difference is equal to or greater than the third threshold value, using the at least one hardware processor.

What is claimed is:

1. A biological information measuring apparatus comprising a sensing apparatus and a calibration device,
the calibration device comprising:
a measuring device configured to intermittently measure first biological information;
first processing circuitry coupled to a first memory, the processing circuitry configured to calculate calibration position information on a position of the calibration device; and
a transmitter configured to transmit the calibration position information and data including the first biological information to the sensing apparatus,
the sensing apparatus comprising:
a receiver configured to receive the first biological information and the calibration position information;
a detector configured to detect a pulse wave continuously in time; and
second processing circuitry coupled to a second memory, the processing circuitry configured to
calibrate the pulse wave based on the first biological information and calculates second biological information based on the calibrated pulse wave,
calculate sensor position information on a position of the sensing apparatus, and
determine whether or not the sensing apparatus and the calibration device are being worn on an identical biological site based on the sensor position information and the calibration position information.

2. The apparatus according to claim 1, wherein each of the first processing circuitry and the second processing circuitry comprises an acceleration sensor or an atmospheric pressure sensor.

3. The apparatus according to claim 2, wherein the second processing circuitry is configured to determine that the sensing apparatus and the calibration device are worn on an identical biological site if a difference in acceleration of the acceleration sensor between the first processing circuitry and the second processing circuitry during a period of time is equal to or below a first threshold value, and if a difference in atmospheric pressure of the atmospheric pressure sensor between the first processing circuitry and the second processing circuitry during the period of time is equal to or below a second threshold value.

4. The apparatus according to claim 1, wherein the second processing circuitry is further configured to
determine whether or not a difference between an amplitude value of a pulse wave detected by the detector before or after a period of time during which the calibration device measures the first biological information, and a minimum amplitude value of the pulse wave detected by the detector during the period of time is equal to or greater than a third threshold value, and
make a notification of incorrect manner of wearing if the difference is determined as being smaller than the third threshold value.

5. The apparatus according to claim 1, wherein
the calibration device further comprises a cuff configured to be worn on a living body during measurement of the first biological information by the measuring device, and configured to expand and contract in volume,
the second processing circuitry is further configured to
determine whether or not a difference between an amplitude value of a pulse wave detected by the detector before or after a period of time during which the calibration device measures the first biological information, and an amplitude value of the pulse wave detected by the detector when the cuff expands to the maximum during the period of time is equal to or greater than a third threshold value, and
make a notification of incorrect manner of wearing if the difference is determined as being smaller than the third threshold value.

6. A biological information measuring apparatus comprising a sensing apparatus and a calibration device,
the calibration device comprising:
a measuring device configured to intermittently measure first biological information; and
a transmitter configured to transmit data including the first biological information,
the sensing apparatus comprising:
a receiver configured to receive the data;
a detector configured to detect a pulse wave continuously in time; and
processing circuitry coupled to a memory, the processing circuitry configured to
determine whether or not a difference between an amplitude value of a pulse wave detected by the detector before or after a period of time during which the calibration device measures the first biological information, and a minimum amplitude value of the pulse wave detected by the detector during the period of time is equal to or greater than a threshold value, and
calibrate the pulse wave based on the first biological information and calculate second biological information based on the calibrated pulse wave if it is determined that the difference is equal to or greater than the threshold value.

7. A biological information measuring apparatus comprising a sensing apparatus and a calibration device,
the calibration device comprising:
a measuring device configured to intermittently measure first biological information;
a cuff configured to be worn on a living body when the measuring device measures the first biological information, and configured to expand and then contract in volume; and
a transmitter configured to transmit data including the first biological information and information on expansion of the cuff,
the sensing apparatus comprising:
a receiver configured to receive the data;
a detector configured to detect a pulse wave continuously in time; and
processing circuitry coupled to a memory, the processing circuitry configured to
determine whether or not a difference between an amplitude value of a pulse wave detected by the detector before or after a period of time during which the calibration device measures the first biological information, and an amplitude value of the pulse wave detected by the detector when the cuff expands to the maximum during the period of time is equal to or greater than a third threshold value, and
calibrate the pulse wave based on the first biological information and calculate second biological information based on the calibrated pulse wave if it is determined that the difference is equal to or greater than the third threshold value.

8. The apparatus according to claim 1, wherein the measuring device is configured to measure the first biological information with higher precision than second biological information obtained from the detector.

9. The apparatus according to claim 6, wherein the measuring device is configured to measure the first biological information with higher precision than second biological information obtained from the detector.

10. The apparatus according to claim 7, wherein the measuring device is configured to measure the first biological information with higher precision than second biological information obtained from the detector.

11. The apparatus according to claim 1, wherein the detector is configured to detect the pulse wave beat by beat, and the first biological information and the second biological information are blood pressures.

12. The apparatus according to claim 6, wherein the detector is configured to detect the pulse wave beat by beat, and the first biological information and the second biological information are blood pressures.

13. The apparatus according to claim 7, wherein the detector is configured to detect the pulse wave beat by beat, and the first biological information and the second biological information are blood pressures.

14. A method of measuring biological information in a biological information measuring apparatus comprising a sensing apparatus and a calibration device, the method comprising:
in the calibration device,
intermittently measuring first biological information;
calculating calibration position information on a position of the calibration device; and
transmitting the calibration position information and data including the first biological information to the sensing apparatus,
in the sensing apparatus,
receiving the first biological information and the calibration position information;
detecting a pulse wave continuously in time;
calibrating the pulse wave based on the first biological information;
calculating second biological information based on the calibrated pulse wave;
calculating sensor position information on a position of the sensing apparatus; and
determining whether or not the sensing apparatus and the calibration device are worn on an identical biological site based on the sensor position information and the calibration position information.

15. A method of measuring biological information in a biological information measuring apparatus comprising a sensing apparatus and a calibration device, the method comprising:
in the calibration device,
intermittently measuring first biological information; and
transmitting data including the first biological information,
in the sensing apparatus,
receiving the data;
detecting a pulse wave continuously in time;
determining whether or not a difference between an amplitude value of a pulse wave detected before or after a period of time during which the calibration device measures the first biological information, and a minimum amplitude value of the pulse wave detected during said period of time is equal to or greater than a threshold value; and
calibrating the pulse wave based on the first biological information and calculating second biological information based on the calibrated pulse wave if it is determined that the difference is equal to or greater than the threshold value.

16. A method of measuring biological information in a biological information measuring apparatus, comprising a sensing apparatus and a calibration device, the method comprising:
in the calibration device,
intermittently measuring first biological information; and
transmitting data including the first biological information and information on expansion of the cuff,
in the sensing apparatus,
receiving the data;
detecting a pulse wave continuously in time;
determining whether or not a difference between an amplitude value of a pulse wave detected before or after a period of time during which the calibration device measures the first biological information, and an amplitude value of the pulse wave detected when the cuff expands to the maximum during said period of time is equal to or greater than a third threshold value; and
calibrating the pulse wave based on the first biological information and calculate second biological information based on the calibrated pulse wave if it is determined that the difference is equal to or greater than the third threshold value.

17. A non-transitory computer readable medium storing a computer program which is executed by a computer to provide the steps of, the computer comprising a sensing apparatus and a calibration device:
intermittently measuring first biological information;
calculating calibration position information on a position of the calibration device;
transmitting the calibration position information and data including the first biological information to the sensing apparatus;
receiving the first biological information and the calibration position information;
detecting a pulse wave continuously in time;
calibrating the pulse wave based on the first biological information;
calculating second biological information based on the calibrated pulse wave;
calculating sensor position information on a position of the sensing apparatus; and
determining whether or not the sensing apparatus and the calibration device are worn on an identical biological site based on the sensor position information and the calibration position information.

18. A non-transitory computer readable medium storing a computer program which is executed by a computer to provide the steps of, the computer comprising a sensing apparatus and a calibration device:
intermittently measuring first biological information;
transmitting data including the first biological information;
receiving the data;
detecting a pulse wave continuously in time;
determining whether or not a difference between an amplitude value of a pulse wave detected before or after a period of time during which the calibration device measures the first biological information, and a minimum amplitude value of the pulse wave detected during said period of time is equal to or greater than a threshold value; and
calibrating the pulse wave based on the first biological information and calculating second biological information based on the calibrated pulse wave if it is determined that the difference is equal to or greater than the threshold value.

19. A non-transitory computer readable medium storing a computer program which is executed by a computer to provide the steps of, the computer comprising a sensing apparatus and a calibration device:
intermittently measuring first biological information;
transmitting data including the first biological information and information on expansion of the cuff;
receiving the data;
detecting a pulse wave continuously in time;
determining whether or not a difference between an amplitude value of a pulse wave detected before or after a period of time during which the calibration device measures the first biological information, and an amplitude value of the pulse wave detected when the cuff expands to the maximum during said period of time is equal to or greater than a third threshold value; and
calibrating the pulse wave based on the first biological information and calculating second biological information based on the calibrated pulse wave if it is determined that the difference is equal to or greater than the third threshold value.
